SEQUENCE LISTING (36 sequences)

SEQ ID NO: 1 - protein, 6 aa, synthetic construct
  SITE 2: note = D-Leucine
  SITE 4: note = D-Phenylalanine
  SITE 6: note = D-Alanine
  REGION 1..6: note = Peptide is circular
TLFFRA

SEQ ID NO: 2 - protein, 6 aa, synthetic construct
  SITE 2: note = D-Leucine
  SITE 4: note = D-Phenylalanine
  SITE 6: note = D-Alanine
  REGION 1..6: note = Peptide is a circular
ALFFRA

SEQ ID NO: 3 - protein, 6 aa, synthetic construct
  SITE 2: note = D-Alanine
  SITE 4: note = D-Phenylalanine
  SITE 6: note = D-Alanine
  REGION 1..6: note = Peptide is circular
TAFFRA

SEQ ID NO: 4 - protein, 6 aa, synthetic construct
  SITE 2: note = D-Leucine
  SITE 4: note = D-Phenylalanine
  SITE 6: note = D-Alanine
  REGION 1..6: note = Peptide is circular
TLAFRA

SEQ ID NO: 5 - protein, 6 aa, synthetic construct
  SITE 2: note = D-Leucine
  SITE 4: note = D-Alanine
  SITE 6: note = D-Alanine
  REGION 1..6: note = Peptide is circular
TLFARA

SEQ ID NO: 6 - protein, 6 aa, synthetic construct
  SITE 2: note = D-Leucine
  SITE 4: note = D-Phenylalanine
  SITE 6: note = D-Arginine
  REGION 1..6: note = Peptide is circular
TLFFAR

SEQ ID NO: 7 - protein, 6 aa, synthetic construct
  SITE 2: note = D-Leucine
  SITE 4: note = D-Phenylalanine
  SITE 6: note = D-Alanine
  REGION 1..6: note = Peptide is circular
KLFFRA

SEQ ID NO: 8 - protein, 6 aa, synthetic construct
  SITE 2: note = D-Leucine
  SITE 4: note = D-Phenylalanine
  SITE 6: note = D-Alanine
  REGION 1..6: note = Pepetide is circular
DLFFRA

SEQ ID NO: 9 - protein, 6 aa, synthetic construct
  SITE 2: note = D-Leucine
  SITE 4: note = D-Phenylalanine
  SITE 6: note = D-Alanine
  REGION 1..6: note = Peptide is circular
VLFFRA

SEQ ID NO: 10 - protein, 6 aa, synthetic construct
  SITE 2: note = D-Aspartic acid
  SITE 4: note = D-Phenylalanine
  SITE 6: note = D-Alanine
  REGION 1..6: note = Peptide is circular
TDFFRA

SEQ ID NO: 11 - protein, 6 aa, synthetic construct
  SITE 2: note = D-Lysine
  SITE 4: note = D-Phenylalanine
  SITE 6: note = D-Alanine
  REGION 1..6: note = Peptide is circular
TKFFRA

SEQ ID NO: 12 - protein, 6 aa, synthetic construct
  SITE 2: note = D-Isoleucine
  SITE 4: note = D-Pheylalanine
  SITE 6: note = D-Alanine
  REGION 1..6: note = Peptide is circular
TIFFRA

SEQ ID NO: 13 - protein, 6 aa, synthetic construct
  SITE 2: note = D-Valine
  SITE 4: note = D-Phenylalanine
  SITE 6: note = D-Alanine
  REGION 1..6: note = Peptide is circular
TVFFRA

SEQ ID NO: 14 - protein, 6 aa, synthetic construct
  SITE 2: note = D-Propargylglycine
  SITE 4: note = D-Phenylalanine
  SITE 6: note = D-Alanine
  REGION 1..6: note = peptide is circular
TXFFRA

SEQ ID NO: 15 - protein, 6 aa, synthetic construct
  SITE 2: note = D-Leucine
  MOD_RES 3: note = 4-Fluorophenylalanine: Phenyl group in phenylalanine is substituted by fluorine at position 4.
  SITE 6: note = D-Alanine
  REGION 1..6: note = Peptide is circular
  SITE 4: note = D-Phenylalanine
TLXFRA

SEQ ID NO: 16 - protein, 6 aa, synthetic construct
  SITE 2: note = D-Leucine
  SITE 4: note = D-Phenylalanine
  SITE 6: note = D-Alanine
  REGION 1..6: note = Peptide is circular
TLYFRA

SEQ ID NO: 17 - protein, 6 aa, synthetic construct
  SITE 2: note = D-Leucine
  SITE 4: note = D-Tyrosine
  SITE 6: note = D-Alanine
  REGION 1..6: note = Peptide is circular
TLFYRA

SEQ ID NO: 18 - protein, 6 aa, synthetic construct
  SITE 2: note = D-Leucine
  MOD_RES 4: note = D-4Fluorophenylalanine: Phenyl group in D-phenylalanine is substituted by fluorine at position 4.
  SITE 6: note = D-Alanine
  REGION 1..6: note = Peptide is circular
TLFXRA

SEQ ID NO: 19 - protein, 6 aa, synthetic construct
  SITE 2: note = D-Leucine
  MOD_RES 4: note = D-3Fluorophenylalanine:Phenyl group in phenylalanine is substituted by fluorine at position 3
  SITE 6: note = D-Alanine
  REGION 1..6: note = Peptide is circular
TLFXRA

SEQ ID NO: 20 - protein, 6 aa, synthetic construct
  SITE 2: note = D-Leucine
  MOD_RES 4: note = 3, 4-difluorophenylalanine. Phenyl group in phenylalanine is substituted by fluorine at positions 3 and 4
  SITE 6: note = D-Alanine
  REGION 1..6: note = Peptide is circular
TLFXRA

SEQ ID NO: 21 - protein, 6 aa, synthetic construct
  SITE 2: note = D-Leucine
  MOD_RES 4: note = 4-benzoylphenylalanine. Phenylalanine is modified at 4th positio with benzoyl group
  SITE 6: note = D-Alanine
  REGION 1..6: note = Peptide is circular
TLFXRA

SEQ ID NO: 22 - protein, 6 aa, synthetic construct
  SITE 2: note = D-Leucine
  SITE 4: note = D-Phenylalanine
  SITE 6: note = D-Serine
  REGION 1..6: note = Peptide is circular
TLFFRS

SEQ ID NO: 23 - protein, 6 aa, synthetic construct
  SITE 2: note = D-Leucine
  SITE 4: note = D-Phenylalanine
  SITE 6: note = D-Diaminopropionic acid
  REGION 1..6: note = Peptide is circular
TLFFRX

SEQ ID NO: 24 - protein, 6 aa, synthetic construct
  SITE 2: note = D-Leucin
  SITE 4: note = D-Phenylalanine
  SITE 5: note = Diaminopropionic acid
  SITE 6: note = D-Arginine
  REGION 1..6: note = Peptide is circular
TLFFXR

SEQ ID NO: 25 - protein, 5 aa, synthetic construct
  SITE 1: note = D-Leucine
  SITE 3: note = D-Phenylalanine
  SITE 5: note = D-Alanine
  REGION 1..5: note = Peptide is circular
LFFKA

SEQ ID NO: 26 - protein, 6 aa, synthetic construct
  MOD_RES 2: note = BODIPY-propargylglycine: D-propargylglycine is conjugated with BODIPY fluorophore.
  SITE 4: note = D-Phenylalanine
  SITE 6: note = D-Alanine
  REGION 1..6: note = Peptide is circular
TXFFRA

SEQ ID NO: 27 - protein, 6 aa, synthetic construct
  SITE 4: note = D-Phenylalanine
  SITE 6: note = D-Arginine
  REGION 1..6: note = Peptide is circular
TVFFAR

SEQ ID NO: 28 - protein, 6 aa, synthetic construct
  SITE 2: note = D-Valine
  SITE 4: note = D-Phenylalanine
  SITE 6: note = D-Arginine
  REGION 1..6: note = Peptide is circular
TVFFAR

SEQ ID NO: 29 - protein, 6 aa, synthetic construct
  SITE 2: note = D-Leucine
  SITE 4: note = D-Phenylalanine
  SITE 6: note = D_Alanine
  REGION 1..6: note = Peptide is circular
TLLFRA

SEQ ID NO: 30 - protein, 6 aa, synthetic construct
  SITE 2: note = D-Leucine
  SITE 4: note = D-Phenylalanine
  SITE 6: note = D-Alanine
  REGION 1..6: note = Peptide is circular
TLRFRA

SEQ ID NO: 31 - protein, 6 aa, synthetic construct
  MOD_RES 3: note = 3-Fluorophenylalanine: Phenyl group in phenylalanine is substituted by fluorine at position 3
  SITE 4: note = D-Phenylalanine
  SITE 6: note = D-Alanine
  REGION 1..6: note = Peptide is circular
  SITE 2: note = D-Leucine
TLXFRA

SEQ ID NO: 32 - protein, 6 aa, synthetic construct
  MOD_RES 3: note = 4-benzoylphenylalanine:Phenylalanine is modified at 4th positio with benzoyl group
  SITE 2: note = D-Leucine
  SITE 4: note = D-Phenylalanine
  SITE 6: note = D-Alanine
  REGION 1..6: note = Peptide is circular
TLXFRA

SEQ ID NO: 33 - protein, 6 aa, synthetic construct
  SITE 2: note = D-Leucine
  SITE 4: note = D-Phenylalanine
  SITE 6: note = D-Propargylglycine
  REGION 1..6: note = Peptide is circular
TLFFRX

SEQ ID NO: 34 - protein, 6 aa, synthetic construct
  SITE 4: note = D-Phenylalanine
  SITE 6: note = D-Arginine
  REGION 1..6: note = Peptide is circular
TAFFAR

SEQ ID NO: 35 - protein, 6 aa, synthetic construct
  SITE 2: note = D-Alanine
  SITE 4: note = D-Phenylalanine
  SITE 6: note = D-Arginine
  REGION 1..6: note = Peptide is circular
TAFFAR

SEQ ID NO: 36 - protein, 6 aa, synthetic construct
  SITE 2: note = D-Alanine
  SITE 4: note = D-Phenylalanine
  SITE 6: note = D-Arginine
  REGION 1..6: note = Peptide is circular
AAFFAR

(19) United States
(12) Patent Application Publication
(10) Pub. No.: US 2025/0073302 A1
(43) Pub. Date: Mar. 6, 2025

(54) CYCLIC PEPTIDES AS PROTEASOME STIMULATORS

(71) Applicant: Purdue Research Foundation, West Lafayette, IN (US)

(72) Inventors: Elizabeth Ivy Parkinson, West Lafayette, IN (US); Darci Jones Trader, West Lafayette, IN (US); Timothy Jonathan Harris, Merrillville, IN (US); Samantha Nelson, West Lafayette, IN (US)

(21) Appl. No.: 18/812,646

(22) Filed: Aug. 22, 2024

Related U.S. Application Data
(60) Provisional application No. 63/536,122, filed on Sep. 1, 2023.

(51) Int. Cl. A61K 38/12

(52) U.S. Cl. CPC A61K 38/12

(57) ABSTRACT

Cyclic peptides that are proteasome stimulators, compositions comprising the same, and their use for stimulating proteasomal degradation of proteins.

### CROSS-REFERENCE TO RELATED APPLICATIONS

This application claims priority to U.S. provisional patent application No. 63/536,122, which was filed Sep. 1, 2023, and which is hereby incorporated by reference in its entirety.

### STATEMENT OF GOVERNMENT SUPPORT

This invention was made with government support under GM138002 awarded by the National Institutes of Health. The government has certain rights in the invention.

### TECHNICAL FIELD

The present disclosure relates to cyclic peptides that stimulate the proteasome and their use for treating a disease or condition caused by an insufficient degradation of proteins by the proteasome system.

### SEQUENCE LISTING

A computer-readable form (CRF) of the Sequence Listing is submitted with this application. The sequence listing is entitled 70287-02_SEQ LISTING.xml, was generated on Aug. 21, 2024 and is 85000 bytes in size. The entire content of the sequence listing incorporated herein by reference in its entirety.

### BACKGROUND

This section introduces aspects that may help facilitate a better understanding of the disclosure. Accordingly, these statements are to be read in this light and are not to be construed as admissions about what is or is not prior art.

One of the most basic, essential cellular processes is the degradation of proteins. The proteasome is a large protein complex responsible for the degradation of intracellular proteins, a process that requires metabolic energy. Protein degradation via the proteasome is done by either a ubiquitin-proteasome system (UPS) or a ubiquitin-independent proteasome system (UIPS). UPS controls almost all basic cellular processes. The proteasome comprises two sub-complexes, 20S core particle (CP) and 19S regulatory particle (RP). The 26S and 30S isoforms of the proteasome, which are composed of the 19S RP and the 20S CP, are responsible for UPS (FIG. 1A). The 20S CP can also degrade proteins alone via UIPS. However, the proteins must be disordered to pass through the gate formed by the α-subunit of the proteasome. Homeostasis between these two pathways, UPS and UIPS, is essential for the maintenance of healthy cells. During the aging process, the production of the 19S RP diminishes, leaving UIPS as the major source of protein degradation. This decline in proteasome activity has been associated with many neurodegenerative diseases, including Huntington's, Alzheimer's, and Parkinson's. These proteinopathies can be caused by the accumulation of misfolded proteins (Abbasbeigi et al., Journal of Chemical Reviews, 2021, 3(1), 97-108). The proteasome can degrade the associated proteins, e.g., α-synuclein and tau (Coleman et al., Chemical Biology, 2021, 2, 636). However, the UIPS is not strongly activated, limiting its ability to clear these toxic proteins.

Various small molecules and a few peptides (FIG. 1B), e.g., AM-404, ursolic acid, miconazole, betulinic acid, chloropromazine, and MK-866, have been found to stimulate the proteasome. However, these proteasome stimulators are disadvantageous due to lack of potency (e.g., AM-404, ursolic acid, and miconazole), selectivity, and inactivity in cell-based assays (e.g., betulinic acid, chloropromazine, and MK-866), thus limiting their translational potential.

Thus, there is an unmet need for a proteasome stimulator that is potent and selective. It is an object of the present disclosure to provide such a proteasome stimulator. This and other objects and advantages, as well as inventive features, will be apparent from the detailed description provided herein.

### SUMMARY

A pharmaceutical composition is provided. The pharmaceutical composition comprises:
(a) a cyclic peptide of formula (I):

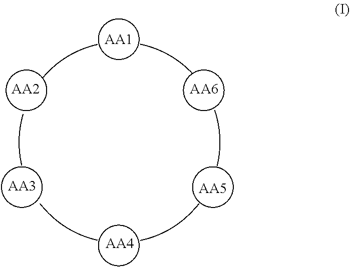

wherein each of AA1, AA2, AA3, AA4, AA5, and AA6 is independently a natural amino acid or an unnatural amino acid with the proviso that formula (I) comprises at least one of each of an aromatic amino acid, a polar amino acid, and an arginine;
and (b) a pharmaceutically acceptable carrier.

The cyclic peptide of formula (I) can comprise six amino acids. The amino acid can be a natural amino acid or an unnatural amino acid. In some embodiments, the natural amino acid or unnatural amino acid is selected from threonine, leucine, phenylalanine, arginine, lysine, aspartic acid, valine, isoleucine, propargylglycine, 3-fluorophenylalanine, 3,4-difluorophenylalanine, tyrosine, 4-benzoylphenylalanine, serine, alanine, 4-fluorophenylalanine, tryptophan, diaminopropionic acid, and diaminobutyric acid.

In some embodiments, the cyclic peptide of formula (I) is CyPPS1, represented by formula (II):

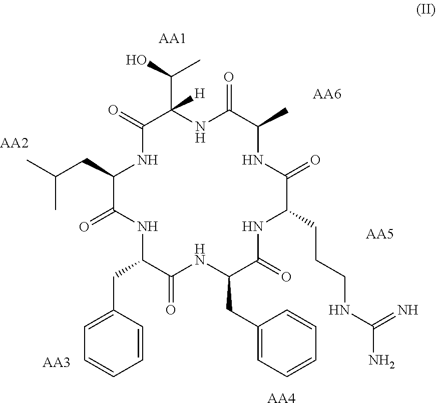

wherein AA1 is Thr, AA2 is D-Leu, AA3 is Phe, AA4 is D-Phe, AA5 is Arg, and AA6 is D-Ala comprising a sequence of [SEQ ID NO: 1] or a derivative thereof selected from: [SEQ ID NO: 2]-[SEQ ID NO: 36].

The pharmaceutical composition can further comprise one or more additional therapeutic agents.

Provided is a method of increasing protein degradation by the proteasome system in a patient with a proteinopathy, which method comprises administering a therapeutically effective amount of a pharmaceutical composition comprising a cyclic peptide of formula (I) and a pharmaceutically acceptable carrier.

In some embodiments, the patient has a prion (misfolded protein) disease, Creutzfeldt-Jakob disease (neurocognitive disorder due to prion disease), Alzheimer's disease, Parkinson's disease, amyloidosis, multiple system atrophy, or Huntington's disease.

A cyclic peptide is provided. In some embodiments, the cyclic peptide is selected from: [SEQ ID NO: 2]-[SEQ ID NO: 36].

### BRIEF DESCRIPTION OF THE DRAWINGS

The present disclosure will be more readily understood from the detailed description of embodiments presented below considered in conjunction with the attached drawings of which:

FIG. 1A shows the structure of the 26S proteasome consisting of 19S regulatory particle (black) and 20S core particle (CP, grey). Ubiquitin-independent proteolysis only consists of the 20S core particle.

FIG. 1B shows the structures of known 20S core particle proteasome stimulators.

FIG. 2A shows 20S CP stimulation of the alanine scan of CyPPS1 by TAS-1 biochemical assay. Two-way analysis of variance (ANOVA) analysis was performed comparing to CyPPS1 (n=3 with SEM indicated) ****P<0.0001, ***P 0.00 1, **P 0.01, *P 0.0 5, and ns P>0.05. Average of three independent replicates.

FIG. 2B shows 20S CP stimulation of CyPPS1, CyPPS8, CyPPS13, CyPPS14, and CyPPS23 stimulators and the liner peptide of CyPPS1 by TAS-1 biochemical assay. Two-way ANOVA analysis was performed comparing to CyPPS1 (n=3 with SEM indicated) ****P<0.0001, ***P 0.001, **P 0.01, *P 0.05, and ns P>0.05. Average of 3 independent replicates.

FIG. 3A shows the dose-response curve of cyclic peptides CyPPS1 CyPPS8, CyPPS13, CyPPS14, and CyPPs23 which are 20S CP stimulators.

FIG. 3B shows quantification of the degradation of highly disordered, e.g., α-synuclein, and low disordered, e.g., glyceraldehyde-3-phosphate dehydrogenase (GAPDH) and lysozyme, proteins. Two-way ANOVA performed comparing to basal activity (n=3 with SEM indicated) ****P<0.0001, ***P<0.001, **P<0.01, *P<0.05, and ns P>0.05. Average of three independent replicates.

FIG. 4A shows Coomassie staining of the degradation of highly disordered a-synuclein and low disordered GAPDH and lysozyme proteins.

FIG. 4B shows the degradation of proteins with varying disorder (α-synuclein, GAPDH, and lysozyme). Lane 1 indicates the latent level, which contained 200 ng of designated protein. Lane 2 was the basal level that was performed with 5 nM 20S CP and 200 ng purified protein. Lanes 3-8 contained 10 uM CyPPS, 5 nM 20S CP, and 200 ng purified protein. CyPPS1 (3), CyPPS23 (4), CyPPS13 (5), CyPPS8 (6), CyPPS14 (7), and PPS1 (8). These data are representative of three separate experiments.

FIG. 5A shows cell permeability, and the mechanism of cell uptake of CyPPSs was determined with 15 μM of CyPPS26 in A549 cells.

FIG. 5B shows flow cytometry analysis of CyPPSs at 10 μM in HEK293 T cells. Two-way ANOVA was performed ((n=3 with SEM indicated) ****P<0.0001, ***P<0.001, **P<0.01, *<0.05, and ns P>0.05. Average of three independent replicates. P<0.05, and ns P>0.05. Average of three independent replicates.

FIG. 6 shows the flow cytometry plots showing shifts in fluorescence signal (FITC) in HEK293 T cells (representative of 3 independent replicates).

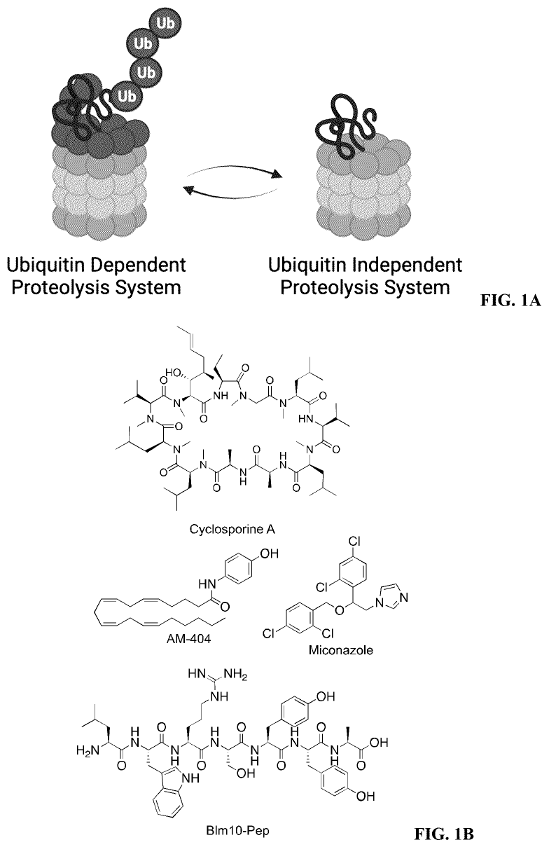

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “proteasome stimulators” refers to the molecules that stimulate proteasomal degradation of disordered proteins while having little to no effect on ordered proteins.

The term “proteinopathy” or “proteopathy” refers to a class of diseases in which certain proteins become structurally abnormal and thereby disrupt the function of cells, tissues, and organs of the body. Proteinopathies include, but are not limited to, neurodegenerative diseases such as prion (misfolded protein) diseases, Creutzfeldt-Jakob disease (neurocognitive disorder due to prion disease), Alzheimer's disease, Parkinson's disease, amyloidosis, multiple system atrophy, Huntington's disease, and a wide range of other disorders.

The term “CyPPS” refers to Cyclic Peptide Proteasome Stimulator.

The term “amino acid” refers to the basic chemical structural unit of a protein or polypeptide. The following abbreviations that follow the standard nomenclature known in the art are used to identify specific amino acids:

| | Three letter | One letter |
|---|---|---|
| Amino Acid | abbreviation | abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Cyclic peptides are more stable than linear peptides and have the ability to enter cells and hit intracellular targets, including proteasomes. The present disclosure is predicted, at least in part, on the discovery that synthetic natural product-inspired cyclic peptides (SNaPP) can be a useful tool for predicting natural product-like molecules for biological testing. Studies reported in Hostetler et al., ACS Chemical Biology, 2021, 2604-2611, revealed that SNaPP was used to generate 51 predicted natural products (pNPs), 14 of which showed antibiotic activity. Thus, SNaPP can be used to synthesize pNPs that can engage in therapeutic activities, such as stimulating 20S core particle (CP) proteasome.

In view of the above, provided is a pharmaceutical composition. The pharmaceutical composition comprises:
(a) a cyclic peptide of formula (I):

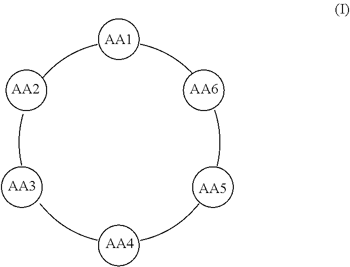

wherein each of AA1, AA2, AA3, AA4, AA5, and AA6 is independently a natural amino acid or an unnatural amino acid with the proviso that formula (I) comprises at least one of each of an aromatic amino acid, a polar amino acid, and an arginine;
and (b) a pharmaceutically acceptable carrier.

Any suitable natural amino acids or unnatural amino acids can be used. The natural amino acid or unnatural amino acid can be selected from threonine (Thr, T), leucine (Leu, L), phenylalanine (Phe, F), arginine (Arg, R), lysine (Lys, K), aspartic acid (Asp, D), valine (Val, V), isoleucine (Ile, I), propargylglycine (Pra), 3-fluorophenylalanine (Phe, 3-F), 3,4-difluorophenylalanine (Phe (3,4-F)), tyrosine (Tyr, Y), 4-benzoylphenylalanine (Bpa), serine (Ser, S), alanine (Ala, A), 4-fluorophenylalanine (Phe, 4-F), tryptophan (Trp, W), diaminopropionic acid (DAP), and diaminobutyric acid (Dab).

The cyclic peptide of formula (I) can be a hexamer, which comprises at least one aromatic amino acid, one polar amino acid, and an arginine that can stimulate proteasomal degradation of the protein. The cyclic peptides containing these amino acids can stimulate the highest percent of the proteasome, for example, about 150% (such as 150%).

In some embodiments, the cyclic peptide of formula (I) is CyPPS1, represented by the formula (II):

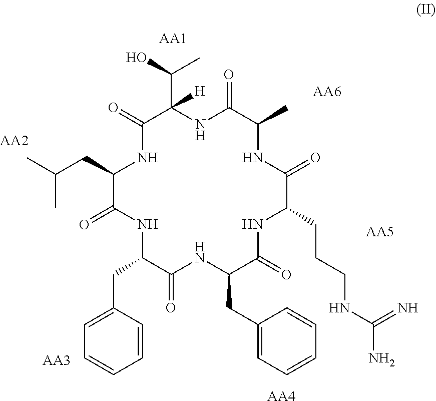

wherein AA1 is Thr, AA2 is D-Leu, AA3 is Phe, AA4 is D-Phe, AA5 is Arg and AA6 is D-Ala comprising a sequence TLFFRA [SEQ ID NO: 1], or a derivative thereof selected from:
(i) CyPPS2: ALFFRA [SEQ ID NO: 2], wherein AA1 is Ala, AA2 is D-Leu, AA3 is Phe, AA4 is D-Phe, AA5 is Arg and AA6 is D-Ala;
(ii) CyPPS3: TAFFRA [SEQ ID NO: 3], wherein AA1 is Thr, AA2 is D-Ala, AA3 is Phe, AA4 is D-Phe, AA5 is Arg and AA6 is D-Ala;
(iii) CyPPS4: TLAFRA [SEQ ID NO: 4], wherein AA1 is Thr, AA2 is D-Leu, AA3 is Ala, AA4 is D-Phe, AA5 is Arg and AA6 is D-Ala;
(iv) CyPPS5: TLFARA [SEQ ID NO: 5], wherein AA1 is Thr, AA2 is D-Leu, AA3 is Phe, AA4 is D-Ala, AA5 is Arg and AA6 is D-Ala;
(v) CyPPS6: TLFFAR [SEQ ID NO: 6], wherein AA1 is Thr, AA2 is D-Leu, AA3 is Phe, AA4 is D-Phe, AA5 is Ala and AA6 is D-Arg;
(vi) CyPPS7: KLFFRA [SEQ ID NO: 7], wherein AA1 is Lys, AA2 is D-Leu, AA3 is Phe, AA4 is D-Phe, AA5 is Arg and AA6 is D-Ala;
(vii) CyPPS8: DLFFRA [SEQ ID NO: 8], wherein AA1 is Asp, AA2 is D-Leu, AA3 is Phe, AA4 is D-Phe, AA5 is Arg and F is D-Ala;
(viii) CyPPS9: VLFFRA [SEQ ID NO: 9], wherein AA1 is Val, AA2 is D-Leu, AA3 is Phe, AA4 is D-Phe, AA5 is Arg and AA6 is D-Ala;
(ix) CyPPS10: TDFFRA [SEQ ID NO: 10], wherein AA1 is Thr, AA2 is D-Asp, AA3 is Phe, AA4 is D-Phe, AA5 is Arg and AA6 is D-Ala;
(x) CyPPS11: TKFFRA [SEQ ID NO: 11], wherein AA1 is Thr, AA2 is D-Lys, AA3 is Phe, AA4 is D-Phe, AA5 is Arg and AA6 is D-Ala;
(xi) CyPPS12: TIFFRA [SEQ ID NO: 12], wherein AA1 is Thr, AA2 is D-Ile, AA3 is Phe, AA4 is D-Phe, AA5 is Arg and AA6 is D-Ala;
(xii) CyPPS13: TVFFRA [SEQ ID NO: 13], wherein AA1 is Thr, AA2 is D-Val, AA3 is Phe, AA4 is D-Phe, AA5 is Arg and AA6 is D-Ala;
(xiii) CyPPS14: TXFFRA [SEQ ID NO: 14], wherein AA1 is Thr, AA2 is D-Pra, AA3 is Phe, AA4 is D-Phe, AA5 is Arg and AA6 is D-Ala;
(xiv) CyPPS15: TLXFRA [SEQ ID NO: 15], wherein AA1 is Thr, AA2 is D-Leu, AA3 is Phe (4F), AA4 is D-Phe, AA5 is Arg and AA6 is D-Ala;
(xv) CyPPS16: TLYFRA [SEQ ID NO: 16], wherein AA1 is Thr, AA2 is D-Leu, AA3 is Tyr, AA4 is D-Phe, AA5 is Arg and AA6 is D-Ala;
(xvi) CyPPS17: TLFYRA [SEQ ID NO: 17], wherein AA1 is Thr, AA2 is D-Leu, AA3 is Phe, AA4 is D-Tyr, AA5 is Arg and AA6 is D-Ala;
(xvii) CyPPS18: TLFXRA [SEQ ID NO: 18], wherein AA1 is Thr, AA2 is D-Leu, AA3 is Phe, AA4 is D-Phe (4F), AA5 is Arg and AA6 is D-Ala;
(xviii) CyPPS19: TLFXRA [SEQ ID NO: 19], wherein AA1 is Thr, AA2 is D-Leu, AA3 is Phe, AA4 is D-Phe (3F), AA5 is Arg and AA6 is D-Ala;
(xix) CyPPS20: TLFXRA [SEQ ID NO: 20], wherein AA1 is Thr, AA2 is D-Leu, AA3 is Phe, AA4 is D-Phe (3, 4F), AA5 is Arg and AA6 is D-Ala;
(xx) CyPPS21: TLFXRA [SEQ ID NO: 21], wherein AA1 is Thr, AA2 is D-Leu, AA3 is Phe, AA4 is D-BPA, AA5 is Arg and AA6 is D-Ala;
(xxi) CyPPS22: TLFFRS [SEQ ID NO: 22], wherein AA1 is Thr, AA2 is D-Leu, AA3 is Phe, AA4 is D-Phe, AA5 is Arg, and AA6 is D-Ser;
(xxii) CyPPS23: TLFFRX [SEQ ID NO: 23], wherein AA1 is Thr, AA2 is D-Leu, AA3 is Phe, AA4 is D-Phe, AA5 is Arg, and AA6 is D-DAP;
(xxiii) CyPPS24: TLFFXR [SEQ ID NO: 24], wherein AA1 is Thr, AA2 is D-Leu, AA3 is Phe, AA4 is D-Phe, AA5 is DAP, and AA6 is D-Arg;
(xxiv) CyPPS26: TXFFRA [SEQ ID NO: 26] wherein AA1 is Thr, AA2 is BODIPY, AA3 is Phe, AA4 is D-Phe, AA5 is Arg, and AA6 is D-Ala;
(xxv) CyPPS27: TVFFAR [SEQ ID NO: 27], wherein AA1 is Thr, AA2 is Val, AA3 is Phe, AA4 is D-Phe, AA5 is Ala, and AA6 is D-Arg;
(xxvi) CyPPS28: TVFFAR [SEQ ID NO: 28], wherein AA1 is Thr, AA2 is D-Val, AA3 is Phe, AA4 is D-Phe, AA5 is Ala, and AA6 is D-Arg;
(xxvii) CyPPS29: TLLFRA [SEQ ID NO: 29], wherein AA1 is Thr, AA2 is D-Leu, AA3 is Leu, AA4 is D-Phe, AA5 is Arg, and AA6 is D-Ala;
(xxviii) CyPPS30: TLRFRA [SEQ ID NO: 30], wherein AA1 is Thr, AA2 is D-Leu, AA3 is Arg, AA4 is D-Phe, AA5 is Arg, and AA6 is D-Ala;
(xxix) CyPPS31: TLXFRA [SEQ ID NO: 31], wherein AA1 is Thr, AA2 is D-Leu, AA3 is Phe (3F), AA4 is D-Phe, AA5 is Arg, and AA6 is D-Ala;
(xxx) CyPPS32: TLXFRA [SEQ ID NO: 32], wherein AA1 is Thr, AA2 is D-Leu, AA3 is BPA, AA4 is D-Phe, AA5 is Arg, and AA6 is D-Ala;
(xxxi) CyPPS33: TLFFRX [SEQ ID NO: 33], wherein AA1 is Thr, AA2 is D-Leu, AA3 is Phe, AA4 is D-Phe, AA5 is Arg, and AA6 is D-Pra;
(xxxii) CyPPS34: TAFFAR [SEQ ID NO: 34], wherein AA1 is Thr, AA2 is Ala, AA3 is Phe, AA4 is D-Phe, AA5 is Ala, and AA6 is D-Arg;
(xxxiii) CyPPS35: TAFFAR [SEQ ID NO: 35], wherein AA1 is Thr, AA2 is D-Ala, AA3 is Phe, AA4 is D-Phe, AA5 is Ala, and AA6 is D-Arg; and
(xxxiv) CyPPS36: AAFFAR [SEQ ID NO: 36], wherein AA1 is Ala, AA2 is D-Ala, AA3 is Phe, AA4 is D-Phe, AA5 is Ala, and AA6 is D-Arg.
   The derivatives CyPPS2 to CyPPS36 are the cyclic derivatives.

Provided is a pharmaceutical composition comprising (i) a cyclic peptide of formula (I), (ii) one or more additional therapeutic agents, and (iii) at least one pharmaceutically acceptable carrier. “Pharmaceutically acceptable carrier” is used herein as an umbrella term to cover the inclusion of carriers, diluents and/or other pharmaceutically acceptable excipients. The carriers, excipients, or diluents can vary based on the particular route of administration (see, e.g., Remington's The Science an Practice of Pharmacy, 23^{rd} ed. (2020)).

Further provided is a method of increasing protein degradation by the proteasome system in a patient with a proteinopathy, which method comprises: administering to the patient a therapeutically effective amount of a pharmaceutical composition comprising a cyclic peptide of formula (I) and a pharmaceutically acceptable carrier. The pharmaceutical composition can further comprise one or more additional therapeutic agents.

In some embodiments, the patient has proteinopathy. Examples of the proteinopathies include, but are not limited to, prion (misfolded protein) diseases, Creutzfeldt-Jakob disease (neurocognitive disorder due to prion disease), Alzheimer's disease, Parkinson's disease, amyloidosis, multiple system atrophy, and Huntington's disease. In some embodiments, the proteinopathy is a neurodegenerative disease.

Provided is a method for treating a proteinopathy in a patient caused by insufficient degradation of proteins by the proteasome system, which method comprises: administering to the patient a therapeutically effective amount of a cyclic peptide of formula (I) or a pharmaceutical composition comprising the same and a pharmaceutically acceptable carrier.

Provided is a cyclic peptide of formula (I):

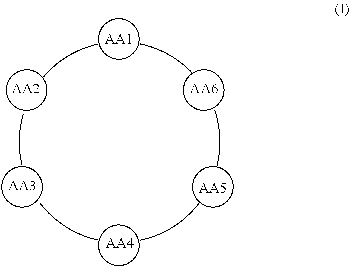

wherein each of AA1, AA2, AA3, AA4, AA5, and AA6 is independently a natural amino acid or an unnatural amino acid with the proviso that formula (I) (a) comprises at least one of each of an aromatic amino acid, a polar amino acid, and an arginine (b) does not comprise the sequence TLFFRA [SEQ ID NO: 1].

The derivatives of CyPPS1, a cyclic peptide of formula (II), can be prepared to increase its potency by increasing cell permeability. Also, the structural activity relationship (SAR) was developed by making derivatives of CyPPS1. An alanine scan was evaluated to determine the amino acid residue that is necessary for the proteasome stimulation activity. The derivatives can be prepared by substituting one or more amino acids. In some embodiments, the derivatives can be prepared by substituting each amino acid of CyPPS1 with alanine except for arginine. The derivative where arginine of AA5 position of CyPPS1 was replaced with alanine was insoluble, so the position of arginine was switched with alanine (e.g., CyPPS6), i.e. D-alanine of AA6 position of CyPPS1 was substituted with D-arginine. When each amino acid was substituted with alanine, the activity decreased, suggesting that all the amino acids are essential for the stimulatory effect.

Provided is a derivative of CyPPS1 of formula (II). In some embodiments, the cyclic peptide derivative is:
CyPPS2: ALFFRA [SEQ ID NO: 2], wherein Thr at AA1 position of CyPPS1 is substituted with Ala;
CyPPS3: TAFFRA [SEQ ID NO: 3], wherein D-Leu at AA2 position of CyPPS1 is substituted with Ala;
CyPPS4: TLAFRA [SEQ ID NO: 4], wherein Phe at AA3 position of CyPPS1 is substituted with Ala;
CyPPS5: TLFARA [SEQ ID NO: 5], wherein D-Phe at AA4 position of CyPPS1 is substituted with D-Ala;
CyPPS6: TLFFAR [SEQ ID NO: 6], wherein Arg at AA5 and D-Ala at AA6 position of CyPPS1are substituted with Ala and D-Arg, respectively;
CyPPS8: DLFFRA [SEQ ID NO: 8], wherein Thr at AA1 position of CyPPS1 is substituted with Asp;
CyPPS13: TVFFRA [SEQ ID NO: 13], wherein D-Leu at AA2 position of CyPPS1 is substituted with D-Val;
CyPPS14: TXFFRA [SEQ ID NO: 14], wherein D-Leu at AA2 position of CyPPS1 is substituted with D-Pra; and
CyPPS23: TLFFRX [SEQ ID NO: 23], wherein D-Ala at AA6 position of CyPPS1 is substituted with D-DAP.

Provided are the other derivatives. The derivatives can be prepared by substituting other amino acids at each position of CyPPS1 (see Table 1). The other amino acids can be acidic, basic, or aliphatic amino acids.

The derivatives prepared are:
CyPPS7: KLFFRA [SEQ ID NO: 7], wherein Thr at AA1 position of CyPPS1 is substituted with Lys;
CyPPS8: DLFFRA [SEQ ID NO: 8], wherein Thr at AA1 position of CyPPS1 is substituted with Asp;
CyPPS9: VLFFRA [SEQ ID NO: 9], wherein Thr at AA1 position of CyPPS1 is substituted with Val;
CyPPS10: TDFFRA [SEQ ID NO: 10], wherein D-Leu at AA2 position of CyPPS1 is substituted with D-Asp;
CyPPS11: TKFFRA [SEQ ID NO: 11], wherein D-Leu at AA2 position of CyPPS1 is substituted with with D-Lys;
CyPPS12: TIFFRA [SEQ ID NO: 12], wherein D-Leu at AA2 position of CyPPS1 is substituted with D-Ile;
CyPPS13: TVFFRA [SEQ ID NO: 13], wherein D-Leu at AA2 position of CyPPS1 is substituted with D-Val;
CyPPS14: TXFFRA [SEQ ID NO: 14], wherein D-Leu at AA2 position of CyPPS1 is substituted with D-Pra;
CyPPS15: TLXFRA [SEQ ID NO: 15], wherein Phe at AA3 position of CyPPS1 is substituted with Phe (4F);
CyPPS16: TLYFRA [SEQ ID NO: 16], wherein Phe at AA3 position of CyPPS1 is substituted with Tyr;
CyPPS17: TLFYRA [SEQ ID NO: 17], wherein D-Phe at AA4 position of CyPPS1 is substituted with Tyr;
CyPPS18: TLFXRA [SEQ ID NO: 18], wherein D-Phe at AA4 position of CyPPS1 is substituted with D-Phe (4F);

CyPPS19: TLFXRA [SEQ ID NO: 19], wherein D-Phe at AA4 position of CyPPS1 is substituted with D-Phe (3F);
CyPPS20: TLFXRA [SEQ ID NO: 20], wherein D-Phe at AA4 position of CyPPS1 is substituted D-Phe (4, 3F);
CyPPS21: TLFXRA [SEQ ID NO: 21], wherein D-Phe at AA4 position of CyPPS1 is substituted with BPA;
CyPPS22: TLFFRS [SEQ ID NO: 22], wherein D-Ala at AA6 position of CyPPS1 is substituted with D-Ser;
CyPPS23: TLFFRX [SEQ ID NO: 23], wherein D-Ala at AA6 position of CyPPS1 is substituted with with D-Dap; and
CyPPS24: TLFFXR [SEQ ID NO: 24], wherein Arg at AA5 position of CyPPS1 is substituted with with D-Dap.

Further, the derivatives of CyPPS1 are provided. The derivatives can be prepared by substituting an amino acid at one or more positions of CyPPS1. In some embodiments, the derivatives are:
CyPPS27: TVFFAR [SEQ ID NO: 27], wherein D-Leu at AA2, Arg at AA5, and D-Ala at AA6 positions of CyPPS1 are substituted with Val, Ala, and D-Arg respectively;
CyPPS28: TVFFAR [SEQ ID NO: 28], wherein D-Leu at AA2 and Arg at AA5 position of CyPPS1 is substituted with Val and Ala respectively;
CyPPS29: TLLFRA [SEQ ID NO: 29], wherein Phe at AA3 position of CyPPS1 is substituted with Leu;
CyPPS30: TLRFRA [SEQ ID NO: 30], wherein Phe at AA3 position of CyPPS1 is substituted with Arg;
CyPPS31, TLXFRA [SEQ ID NO: 31], wherein Phe at AA3 position of CyPPS1 is substituted with Phe (3F);
CyPPS32: TLXFRA [SEQ ID NO: 32], wherein Phe at AA3 position of CyPPS1 is substituted with BPA;
CyPPS33: TLFFRX [SEQ ID NO: 33], wherein D-Ala at AA6 position of CyPPS1 is substituted with D-Pra;

CyPPS34: TAFFAR [SEQ ID NO: 34], wherein D-Leu at AA2, Arg at AA5 and D-Ala at AA6positions of CyPPS1 are substituted with Ala, Ala, and D-Arg respectively;
CyPPS35: TAFFAR [SEQ ID NO: 35], wherein D-Leu at AA2, Arg at AA5 and D-Ala at AA6 positions of CyPPS1 are substituted with D-Ala, Ala and D-Arg respectively;
CyPPS36: AAFFAR [SEQ ID NO: 36], wherein Thr at AA1, D-Leu at AA2, Arg at AA5 and D-Ala at AA6 positions of CyPPS1 are substituted with Ala, D-Ala, Ala and D-Arg respectively; and
CyPPS26, a flurophore conjugation: TXFFRA [SEQ ID NO: 26], wherein AA1 is Thr, AA2 is BODIPY, AA3 is Phe, AA4 is D-Phe, AA5 is Arg, and AA6 is D-Ala.

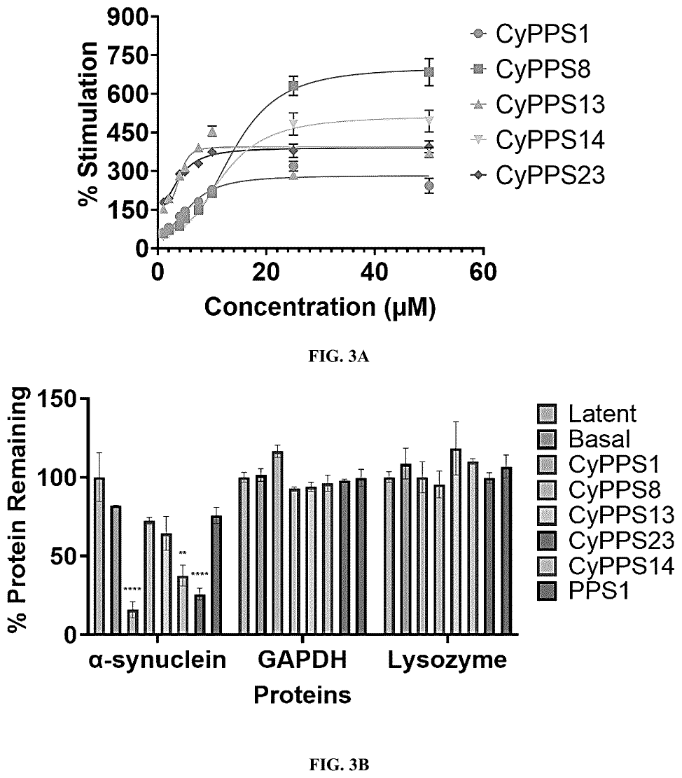

Based on the potency of CyPPSs in the TAS-1 assay, their ability to degrade proteins was tested using an in vitro protein degradation assay. In vitro protein degradation assays revealed that these molecules can stimulate proteasomal degradation of disordered proteins (e.g., α-synuclein), while having little to no effect on ordered proteins (e.g., lysozyme and GAPDH). Cyclic peptides, for example, CyPPS1, CyPPS14, and CyPPS23, greatly enhanced proteasomal degradation of α-synuclein, while having little-to-no effect on GAPDH and lysozyme. These cyclopeptides were also tested for toxicity to mammalian cells. No toxicity was observed in HEK293 cells. Additionally, no hemolysis was observed with human red blood cells, further supporting these as excellent lead molecules (see Table 3). The dose-response relationship was determined for the top five cylcopeptides CyPPs1, CyPPS8, CyPPS13, CyPPS23, and CyPPS14. FIG. 3A illustrates that CyPPS23 and CyPPS13 are the most potent compounds with the lowest EC₅₀ values (4.0 μM and 4.1 μM, respectively). However, their maximal response was lower compared to CyPPS8 and CyPPS14 (see Table 2).

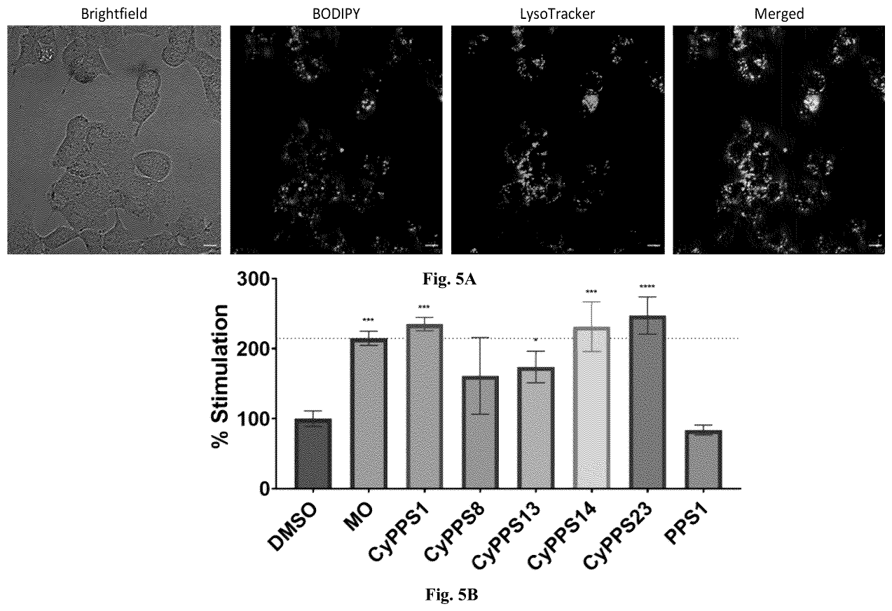

Further, the ability of cyclopeptides to enter the cells was studied. CyPPS26, a BODIPY-tagged CyPPS, was synthesized from the alkyne derivative CyPPS14 using BODIPY™ FL azide dye (Scheme 2). A549 cells were then dosed with CyPPS26 and analyzed for cell permeability via confocal microscopy. Further investigated was the mechanism by which CyPPS26 was entering the cell. Cellular uptake of cyclic peptides usually occurs either via passive diffusion or endocytosis. The puncta present suggests that CyPPSs likely enter the cell via endosomal uptake. Endosomal uptake was confirmed by the overlap of the BODIPY™ and LysoTracker signal (FIG. 5A). The cytosolic fluorescence observed suggests that CyPPSs can escape the endosome, and thus likely can engage with the cytosolic proteasome. Cytosolic protein accumulation is linked to many neurodegenerative diseases, including α-synuclein accumulation in Parkinson's disease. Given that CyPPSs can degrade highly disordered proteins and are cell-permeable, their abilities to stimulate the proteasome in cellulo using a flow cytometry assay was studied.

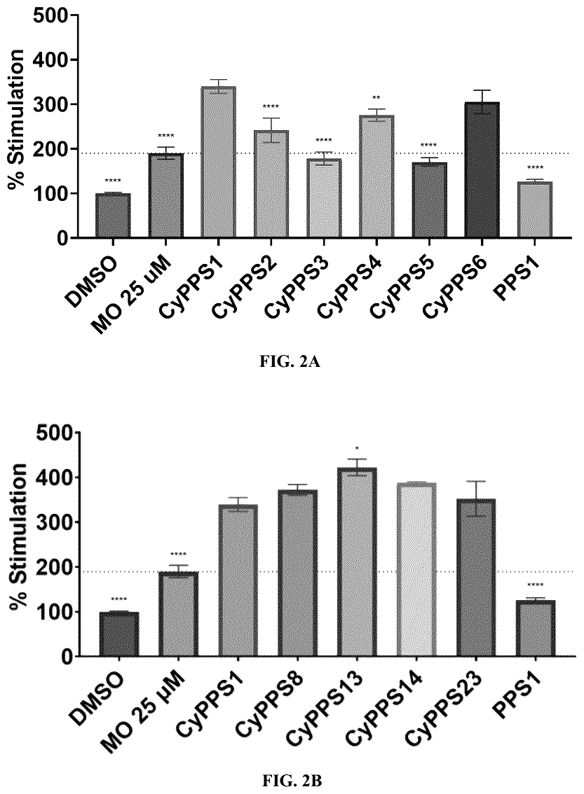

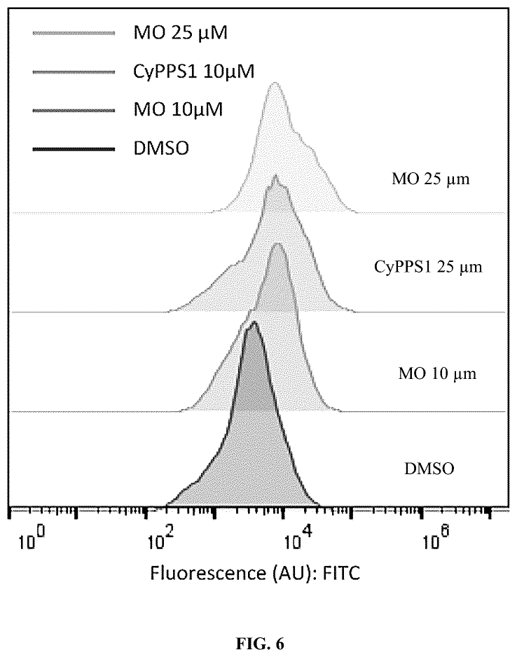

Flow cytometry allows for the study of proteasomal activity in physiologically relevant conditions and presents a high-throughput alternative to traditional gel electrophoresis and western blotting techniques. To test the assay, the covalent fluorescent-based probe was applied in HEK293 T cells to quantify proteasome stimulation of CyPPSs alone and in the presence of the known proteasome stimulator miconazole and the known proteasome inhibitor MG132. The covalent fluorescent-based probe utilized an epoxy-ketone warhead that interacts with the β5 subunit of the proteasome, forming a covalent attachment and fluorescently tagging the proteasome. The addition of the CyPPSs induces a significant shift in the intracellular fluorescence (FIG. 6). This shift validated that CyPPSs can stimulate cytosolic proteasome in cellulo. Interestingly, the same compounds that degrade purified α-synuclein (CyPPS1, CyPPS14, and CyPPS23) showed significant cellular stimulation of the proteasome. However, their activity was lower than what would be expected based on their in vitro activity. This may be due to incomplete cellular uptake or escape from the endosomes. The PPS1, a linear peptide of CyPPS1, does not stimulate the proteasome. This is likely due to a combination of effects, including the inability to stimulate the proteasome (FIGS. 2A and 2B) as well as the generally poor cell permeability and stability of linear peptides.

Cyclic peptides can be synthesized in accordance with methods known in the art, such as solid-phase peptide synthesis.

### Schemes

Scheme 1: illustrates the synthesis of cyclopeptides (CyPPSs)

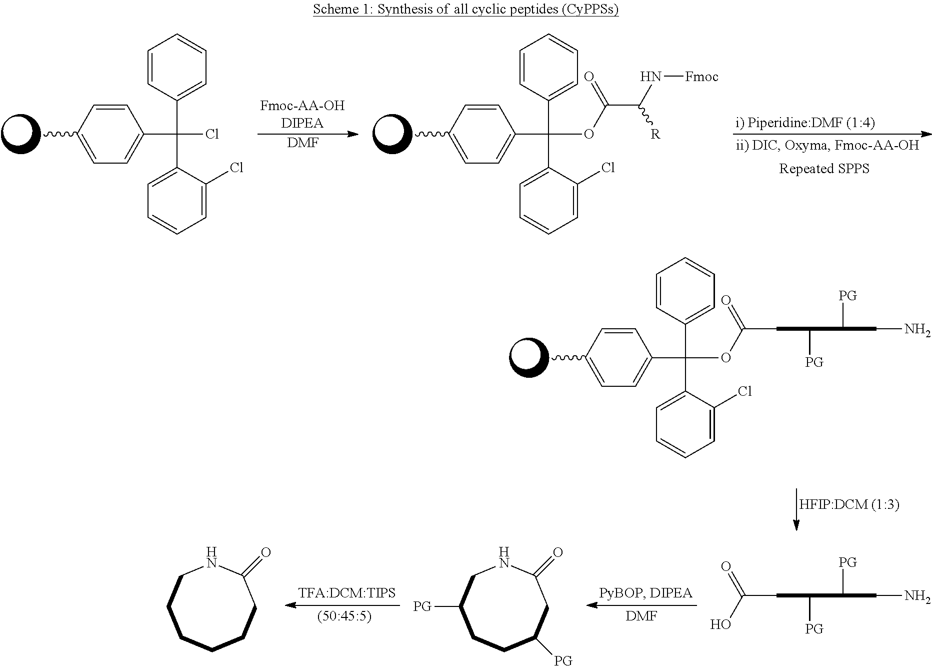

All CyPPSs were synthesized with solid-phase peptide synthesis (SPPS) on 2-chlorotrityl chloride resin (2-CTC). Fluorenylmethoxycarbonyl (Fmoc) removal was performed with 20% piperidine in dimethylformamide (DMF). Amide coupling of the amino acids was done with N,N′-diisopropylcarbodiimide (DIC), and oxyma. The linear peptide on 2-CTC resin was removed from the resin with hexafluoro-2-propanol (HFIP). The linear peptide was cyclized using benzotriazole-1-yloxytripyrrolidinophosphonium hexafluorophosphate (PyBOP), and the removal of protecting groups by trifluoroacetic acid (TFA) yielded the final cyclic peptide.

### Scheme 2:

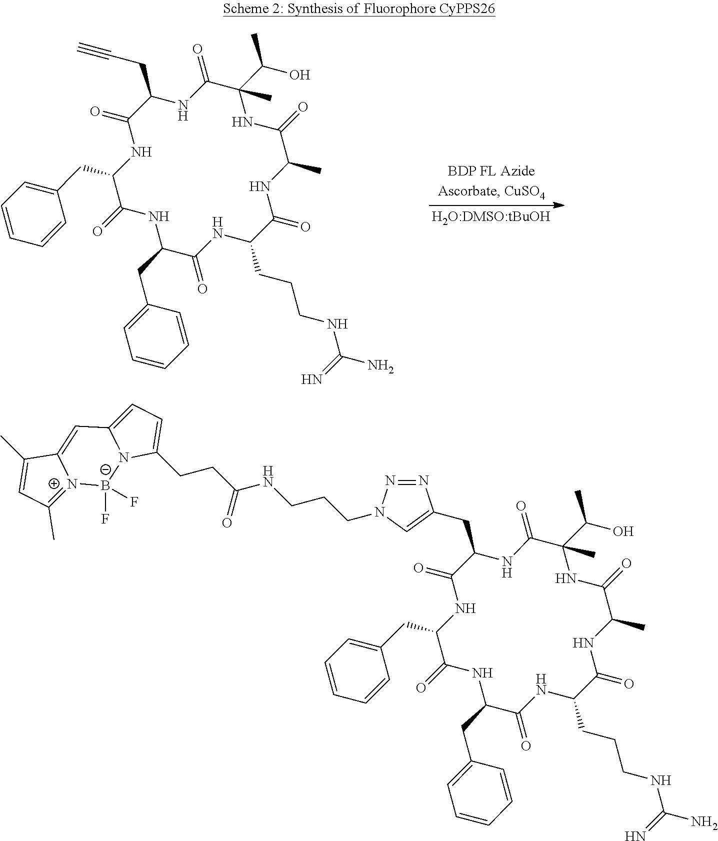

CyPPS14 (7.35 μmol) was dissolved in 600 μL tert-butanol. Copper sulfate (0.68 μmol) and ascorbate (6.66 μmol) in 80 μL of water were added to CyPPS14 along with 160 μL BODIPY™ FL azide (6.68 μmol) in dimethylsufoxide (DMSO). The mixture was sonicated for 5 minutes, capped with argon, and stirred for 1 hour at 80° C. Purification and identification of CyPPS26 (28%) was performed by high-performance liquid chromatography (HPLC) and mass spectrometry (MS).

The term “amino acid” generally refers to an organic compound comprising both a carboxylic acid group and an amine group. The term “amino acid” includes both “natural” and “unnatural” or “non-natural” amino acids. Additionally, the term amino acid includes O-alkylated or N-alkylated amino acids, as well as amino acids having nitrogen or oxygen-containing side chains (such as Lys, Orn, or Ser) in which the nitrogen or oxygen atom has been acylated or alkylated. Amino acids may be pure L or D isomers or mixtures of L and D isomers, including racemic mixtures.

The term “natural amino acid” and equivalent expressions refer to L-amino acids commonly found in naturally occurring proteins. Examples of natural amino acids include, without limitation, alanine (Ala), cystein (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asp), proline (Pro), glutamine (Gin), arginine (Arg), serine (Ser), threonine (Thr), valine (Val), tryptophan (Trp), tyrosine (Tyr), β-alanine (β-ALA), and γ-aminobutyric acid (GABA).

The term “unnatural amino acid” refers to any derivative of a natural amino acid including D-forms and α-and β-amino acid derivatives. The terms “unnatural amino acid” and “non-natural amino acid” are used interchangeably herein and are meant to include the same moieties. It is noted that certain amino acids, e.g., hydroxyproline, that are classified as a non-natural amino acid herein, may be found in nature within a certain organism or a particular protein. Amino acids with many different protecting groups appropriate for immediate use in the solid-phase synthesis of peptides are commercially available.

The term “therapeutically effective amount” or “therapeutically effective dose” refers to an amount of the active ingredient(s) that is(are) sufficient, when administered, to deliver efficaciously the active ingredient(s) for the treatment of a disease or condition of interest to a subject in need thereof. The term “prophylactically effective amount” refers to an amount to prevent or postpone the development of a disease or condition, suppress symptoms that may appear, or reduce the risk of developing or recurrence of a disease or condition. The prophylactically or therapeutically effective amount of such composition will vary depending upon the patient and the disease or condition being treated, the weight and age of the patient, the severity of the disease or condition, the manner of administration, and the like, which can readily be determined by one of ordinary skill in the art. In the case of a cancer or other proliferative disorder, the prophylactically or therapeutically effective amount of the agent may reduce (i.e., inhibit to some extent or stop) unwanted cellular proliferation; reduce the number of cancer cells; reduce the tumor size; inhibit (or stop) cancer cell infiltration into peripheral organs; inhibit (or stop) tumor metastasis; inhibit, e.g., to some extent, tumor growth; and/or relieve, to some extent, one or more of the signs or symptoms associated with the cancer. To the extent the administered compound or composition prevents growth and/or kills existing cancer cells, it may be cytostatic and/or cytotoxic.

For any compound or composition, a therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a prophylactic or therapeutically effective amount of a combination or composition described herein to a subject in need of such treatment. The treatment can be orientated symptomatically, for example, to suppress symptoms. It can be effected over a short period, be oriented over a medium term, or can be a long-term treatment, for example within the context of maintenance therapy.

Generally, daily oral doses of a compound are from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug level, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

The compounds can be typically administered in admixture with a pharmaceutical carrier to give a pharmaceutical composition selected with regard to the intended route of administration and standard pharmaceutical practice. Pharmaceutical compositions can be formulated in a conventional manner using one or more physiologically acceptable carriers comprising excipients and/or auxiliaries that facilitate the processing of the compound. The pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder can contain about 0.01% to about 95%, and preferably from about 1% to about 50%, of the combination of compounds. When administered in liquid form, a liquid carrier can be added, such as water, petroleum, or oils of animal or plant origin. The liquid form of the composition can further contain the physiological saline solution, dextrose or other saccharide solutions, or glycols. When administered in liquid form, the composition contains about 0.1% to about 90%, and preferably about 1% to about 50%, by weight, of the combination of compounds. For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

The exact formulation, route of administration, and dosage of a pharmaceutical composition comprising an effective amount of the compound are determined by an individual physician in view of the diagnosed condition or disease. The dosage amount and interval can be adjusted individually to provide levels of the compound that are sufficient to maintain a prophylactic or therapeutic effect.

Toxicity and therapeutic efficacy of the combination can be determined by standard pharmaceutical procedures in cell cultures or experimental animals, e.g., for determining the maximum tolerated dose (MTD) of a compound, which is defined as the highest dose that causes no toxicity in animals. The therapeutic index is the dose ratio between the maximum tolerated dose and therapeutic effects (e.g., inhibition of tumor growth). The dosage can vary within this range depending upon the dosage form employed and the route of administration utilized. The determination of a therapeutically effective amount is well within the capability of those ordinarily skilled in the art, especially in light of the detailed disclosure provided herein.

A combination can be administered in an amount of about 0.005 to about 500 milligrams per dose, about 0.05 to about 250 milligrams per dose, or about 0.5 to about 100 milligrams per dose. For example, the combination can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

As stated above, a combination or composition described herein can be administered in with one or more other prophylactically or therapeutically active agents.

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

### EXAMPLES

The following examples serve to illustrate the present disclosure. The examples are not intended to limit the scope of the claimed invention in any way.

### Biochemical Assay

### SNaPP 20S CP Stimulators

A library of 45 cyclic peptides were screened, that were generated by SnaPP by utilizing the TAS-1 biochemical assay. In the library, nine peptides that stimulated the proteasome 20% more than the DMSO standard gave a hit rate of 20%. This was in stark contrast to many other proteasome stimulator screens, which typically have hit rates of less than 1%. Of the hits identified in the initial screen, six stimulated the proteasome greater than 140%. These top compounds all contained an arginine, a polar-uncharged amino acid, and an aromatic amino acid. Furthermore, four of the six with the highest percent stimulation contained six amino acids. Analogous to the peptides in many peptide proteasome stimulators have an arginine. Another common motif throughout peptide stimulators is a tyrosine that has shown to open or stabilize the gate of the 20S CP. However, none of the top pNP stimulators have tyrosine but do have a conserved phenylalanine, which may be acting similarly.

### Derivative Preparation

It was seen that pNP-40 (renamed CyPPS1) has the highest percent stimulation. The derivatives of CyPPS1 was prepared for a structural activity relationship (SAR) (Table 1). An alanine scan was first performed to determine the residues that were necessary for activity. Each amino acid, except for arginine, was substituted with an alanine. Unfortunately, the derivative where arginine was replaced with alanine was insoluble and thus could not be evaluated. For this reason, a derivative was prepared that switched the position of arginine with alanine (CyPPS6). For this derivative, the stimulatory activity was preserved, suggesting that the position of the arginine was not essential. When each amino acid was substituted with an alanine the activity decreased, suggesting that all of the amino acids are essential for the stimulatory effect (FIG. 2A). Specifically, replacing either the D-leucine (CyPPS3) or the D-phenylalanine (CyPPS5) with D-alanine resulted in the greatest loss of stimulatory activity, suggesting that these amino acids are particularly important for activity. Additionally, a linear version of CyPPS1 (PPS1) was explored and found to have little-to-no stimulatory effect. This provides strong evidence for the necessity of cyclizing these peptides for activity and suggests that cyclization likely holds them in active conformation.

The SAR (Table 1) was further explored by substituting other amino acids at each position. The threonine at position I was substituted with lysine (CyPPS7), aspartic acid (CyPPS8), and valine (CyPPS9). The lysine substitution decreased activity, suggesting basic amino acids are not tolerated at position 1. Both CyPPS8 and CyPPS9 maintained stimulatory activity, suggesting that position 1 tolerates both acidic and aliphatic amino acids, in addition to the polar threonine. The D-leucine at position 2 was substituted with D-aspartic acid (CyPPS10), D-lysine (CyPPS11), D-isoleucine (CyPPS12), and D-valine (CyPPS13). Only CyPPS13 retained activity, with the other derivatives having decreased activity. There was the lack of activity with CyPPS12. These results overall suggested that an aliphatic amino acid with branching was preferred. Position 2 was also substituted with D-propargylglycine (CyPPS14), which also retained stimulatory activity, supporting the necessity for an aliphatic side chain. All substitutions at position 3 decreased activity except for 4-fluorophenylalanine (CyPPS15). Tyrosine (CyPPS16/CyPPS17) was also substituted for position 3 and 4. Interestingly, a decline in stimulatory activity was observed, indicating these pNPs are likely not acting via the same mechanism as the previously studied tyrosine-containing peptides. Substitution of D-4-fluorophenylalanine (CyPPS18) at position 4 decreased activity. Other substitutions, such as D-3-flurophenylalanine (CyPPS19) and D-3,4-difluorophenylalanine (CyPPS20) retained activity, suggesting that electron-withdrawing groups are tolerated. This position was also substituted for 4-benzoylphenylalanine (CyPPS21), and found that the 20S CP stimulatory activity was maintained. Substitution of the D-alanine at position 6 with D-serine (CyPPS22) and D-diaminopropionic acid (CyPPS23) maintained stimulatory activity, suggesting that small polar substitutions are tolerated. It was observed that position 5 can not be substituted with any amino acids other than basic amino acids due to solubility issues. The basic amino acids, such as diaminopropionic acid (CyPPS24) maintained stimulatory activity. Finally, a cyclic pentapeptide (CyPPS24) was explored but had decreased activity, demonstrating the importance of ring size for activity. Of the 37 derivatives tested, 4 derivatives were found to have similar or increased stimulatory activity compared to the parent CyPPS1 (FIG. 2B).

TABLE 1
CyPPS derivatives

| | | [M + H]+ | | | | HPLC | |
|---|---|---|---|---|---|---|---|
| Cyclic | | (formula, | Yield | | Purifi- | % | Peak |
| Peptides | Structure | calcd) | % | mg | cation | Purity | (min) |
| CyPPS1 | TLFFRA [SEQ ID NO: 1] | 736.4, | 32 | 13.5 | A1, B1 | >99 | 10.9 |
| | | 736.6 | | | | | |
| CyPPS2 | ALFFRA [SEQ ID NO: 2] | 706.9, | 20 | 6.92 | A1, B1 | 98 | 10.6 |
| | | 706.4 | | | | | |
| CyPPS3 | TAFFRA [SEQ ID NO: 3] | 694.4, | 17 | 6.39 | A1, B1 | >99 | 11.6 |
| | | 694.1 | | | | | |
| CyPPS4 | TLAFRA [SEQ ID NO: 4] | 660.4, | 4 | 1.48 | A1, B2 | 91 | 8.9 |
| | | 660.6 | | | | | |
| CyPPS5 | TLFARA [SEQ ID NO: 5] | 660.4, | 42 | 16.44 | A1, B2 | >99 | 10.3 |
| | | 661.2 | | | | | |
| CyPPS6 | TLFFAR [SEQ ID NO: 6] | 736.4, | 37 | 13.63 | A1, B2 | >99 | 11.8 |
| | | 737.4 | | | | | |
| CyPPS7 | KLFFRA [SEQ ID NO: 7] | 763.4, | 33 | 12.53 | A1, B1 | 96 | 8.8 |
| | | 763.1 | | | | | |
| CyPPS8 | DLFFRA [SEQ ID NO: 8] | 750.4, | 58 | 21.64 | A1, B1 | 96 | 11.2 |
| | | 750.5 | | | | | |
| CyPPS9 | VLFFRA [SEQ ID NO: 9] | 734.4, | 38 | 12.91 | A1, B1 | 98 | 14.2 |
| | | 734.2 | | | | | |
| CyPPS10 | TDFFRA [SEQ ID NO: 10] | 738.4, | 88 | 32.44 | A1, B1 | 92 | 8.4 |
| | | 737.2 | | | | | |
| CyPPS11 | TKFFRA [SEQ ID NO: 11] | 751.4, | 21 | 8.07 | A1, B1 | 91 | 6.1 |
| | | 750.8 | | | | | |
| CyPPS12 | TIFFRA [SEQ ID NO: 12] | 736.9, | 11 | 3.92 | A2, B1 | 99 | 10.1 |
| | | 736.5 | | | | | |
| CyPPS13 | TVFFRA [SEQ ID NO: 13] | 722.4, | 30 | 11.33 | A1, B1 | 96 | 10.6 |
| | | 722.4 | | | | | |
| CyPPS14 | TXFFRA [SEQ ID NO: 14] | 718.5, | 49 | 17.56 | A1, B2 | 92 | 10.3 |
| | | 718.4 | | | | | |
| CyPPS15 | TLXFRA [SEQ ID NO: 15] | 754.4, | 24 | 9.1 | A1, B1 | 97 | 11.5 |
| | | 754.8 | | | | | |
| CyPPS16 | TLYFRA [SEQ ID NO: 16] | 752.4, | 15 | 5.68 | A1, B1 | 94 | 9.4 |
| | | 753.2 | | | | | |
| CyPPS17 | TLFYRA [SEQ ID NO: 17] | 752.4, | 2 | 0.88 | A1, B2 | >99 | 10.5 |
| | | 751.2 | | | | | |
| CyPPS18 | TLFXRA [SEQ ID NO: 18] | 754.4, | 14 | 5.39 | A1, B1 | 95 | 11.9 |
| | | 755.3 | | | | | |
| CyPPS19 | TLFXRA [SEQ ID NO: 19] | 754.4, | 16 | 5.99 | A1, B1 | >99 | 11.7 |
| | | 755.3 | | | | | |
| CyPPS20 | TLFXRA [SEQ ID NO: 20] | 772.4, | 12 | 4.75 | A1, B1 | 96 | 11.4 |
| | | 773.4 | | | | | |
| CyPPS21 | TLFXRA [SEQ ID NO: 21] | 841.0, | 36 | 15.03 | A1, B1 | 97 | 11.9 |
| | | 840.4 | | | | | |
| CyPPS22 | TLFFRS [SEQ ID NO: 22] | 752.4, | 21 | 7.86 | A1, B1 | 98 | 12.6 |
| | | 753.4 | | | | | |
| CyPPS23 | TLFFRX [SEQ ID NO: 23] | 751.4, | 24 | 8.98 | A1, B1 | >99 | 8.9 |
| | | 752.4 | | | | | |
| CyPPS24 | TLFFXR [SEQ ID NO: 24] | 751.4, | 95 | 37.52 | A1, B1 | 91 | 9 |
| | | 751.4 | | | | | |
| CyPPS25 | LFFKA [SEQ ID NO: 25] | 607.4, | 71 | 30.34 | A1, B1 | >99 | 11.4 |
| | | 607.2 | | | | | |
| CyPPS27 | TVFFAR [SEQ ID NO: 27] | 722.4, | 28 | 10.03 | A2, B1 | 93 | 9.2 |
| | | 722.4 | | | | | |
| CyPPS28 | TVFFAR [SEQ ID NO: 28] | 722.4, | 48 | 16.6 | A1, B1 | 96 | 10.8 |
| | | 722.4 | | | | | |
| CyPPS29 | TLLFRA [SEQ ID NO: 29] | 546.3, | 2 | 0.42 | A1, B1 | 92 | 14.8 |
| | | 547.0 | | | | | |
| CyPPS30 | TLRFRA [SEQ ID NO: 30] | 775.4, | 3 | 1.11 | A1, B1 | 94 | 10.2 |
| | | 776.2 | | | | | |
| CyPPS31 | TLXFRA [SEQ ID NO: 31] | 754.4, | 30 | 11.43 | A1, B1 | 99 | 12.8 |
| | | 753.8 | | | | | |
| CyPPS32 | TLXFRA [SEQ ID NO: 32] | 841.0, | 30 | 12.51 | A1, B2 | 95 | 11.3 |
| | | 840.4 | | | | | |
| CyPPS33 | TLFFRX [SEQ ID NO: 33] | 760.5, | 66 | 25.16 | A1, B1 | 90 | 12.5 |
| | | 760.4 | | | | | |
| CyPPS34 | TAFFAR [SEQ ID NO: 34] | 694.4, | 26 | 9.56 | A2, B1 | 98 | 9.1 |
| | | 694.4 | | | | | |
| CyPPS35 | TAFFAR [SEQ ID NO: 35] | 694.4, | 77 | 37.54 | A1, B1 | 94 | 9.8 |
| | | 694.2 | | | | | |
| CyPPS36 | AAFFAR [SEQ ID NO: 36] | 664.4, | 61 | 20.2 | A1, B1 | 94 | 9.6 |
| | | 664.4 | | | | | |

“A” is purification after resin cleavage and
“B” is purification after TFA deprotection.
A1: Precipitation in 10 mL of 1:1 acetonitrile:water.
A2: Preporatory HPLC with a gradient of 0-1 minutes 5% acetonitrile (95% water, 0.1% formic acid), 1-20 minutes (5-95% acetonitrile), 20-25 minutes 5% acetonitrile.
B1: precipitation in 10 mL of MTBE.
B2: Preparatory HPLC with a gradient of 0-1 minutes 5% acetonitrile (95% water, 0.1% formic acid), 1-20 minutes (5-95% acetonitrile), 20-25 minutes 5% acetonitrile.

### Dose-Response Curve

The dose-response relationship was determined for the top five hits (FIG. 3A). CyPPS23 and CyPPS13 had the lowest EC values (4.1 μM and 4.0 μM, respectively), suggesting they are the most potent compounds. However, their maximal response (Eₘₐₓ 391 and 393, respectively) was lower compared to CyPPS8 and CyPPS14 (see Table 2). The structural similarity of the molecules suggested that they likely can have similar binding sites.

TABLE 2
Dose-Response Curve

| | | EC50 | |
|---|---|---|---|
| Name | SEQ ID | (μM) | Emax |
| CyPPS1 | SEQ ID NO: 1 | 6.4 | 282.9 |
| CyPPS8 | SEQ ID NO: 8 | 14.2 | 701.4 |
| CyPPS13 | SEQ ID NO: 13 | 4 | 393.8 |
| CyPPS14 | SEQ ID NO: 14 | 12.1 | 513.6 |
| CyPPS23 | SEQ ID NO: 23 | 4.1 | 390.9 |

### Purified Protein Degradation

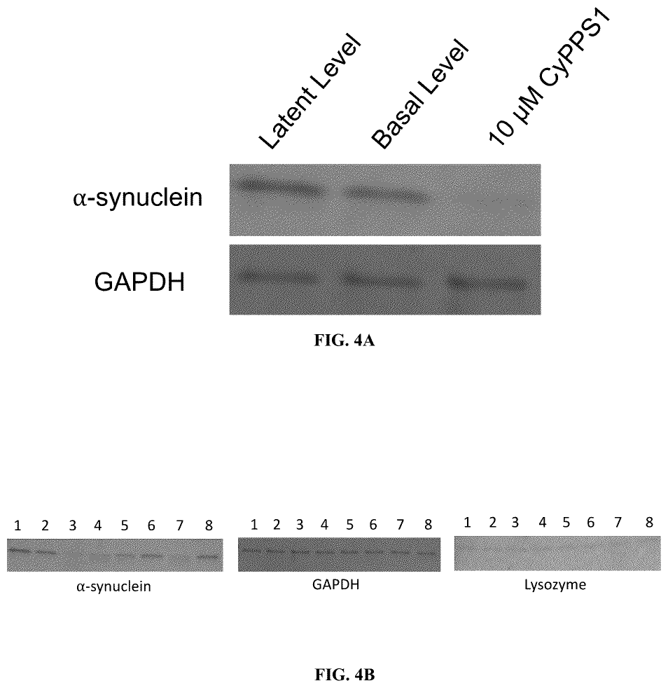

Given the potency of the CyPPSs in the TAS-1 assay, their abilities to degrade proteins using an in vitro degradation assay was explored. The 20S CP typically degrades proteins that are highly disordered. To ensure the selectivity of the CyPPS for disordered proteins, the ability of these molecules to induce degradation of disordered proteins (e.g. α-synuclein) and ordered proteins (e.g., GAPDH and lysozyme) was explored. CyPPS1, CyPPS14, and CyPPS 23 greatly enhanced proteasomal degradation of α-synuclein while having little-to-no effect on GAPDH and lysozyme (FIGS. 3B and 4A). Overall, this suggests that CyPPS1, CyPPS14, and CyPPS 23 are excellent leads for proteasome stimulation. The top hits were also tested for toxicity to mammalians cells. No toxicity was observed with either HEK293 or A549 cell lines (see Table 3). Additionally, no hemolysis was observed with human red blood cells, further supporting these as excellent lead molecules.

TABLE 3
Biochemical Assays (TAS-1, Cytotoxicity (15 uM))

| Cyclic | | | | HEK | Hemo- |
|---|---|---|---|---|---|
| Peptide | Sequence ID | TAS-1 | In-Cellulo | 293 | lysis |
| CyPPS1 | SEQ ID NO: 1 | 339.5 ± 15.7 | 235.1 ± 5.5 | 0 ± 15 | 13 ± 1 |
| CyPPS2 | SEQ ID NO: 2 | 241.3 ± 27.6 | — | — | — |
| CyPPS3 | SEQ ID NO: 3 | 177.9 ± 14.7 | — | — | — |
| CyPPS4 | SEQ ID NO: 4 | 275.1 ± 14.0 | — | — | — |
| CyPPS5 | SEQ ID NO: 5 | 170.3 ± 9.6 | — | — | — |
| CyPPS6 | SEQ ID NO: 6 | 304.9 ± 26.3 | — | — | — |
| CyPPS7 | SEQ ID NO: 7 | 225.9 ± 6.0 | — | — | — |
| CyPPS8 | SEQ ID NO: 8 | 372.2 ± 12.1 | 161.0 ± 31.7 | 0 ± 16 | 0 ± 0 |
| CyPPS9 | SEQ ID NO: 9 | 310.2 ± 5.1 | — | — | — |
| CyPPS10 | SEQ ID NO: 10 | 173.4 ± 6.3 | — | — | — |
| CyPPS11 | SEQ ID NO: 11 | 100.8 ± 5.9 | — | — | — |
| CyPPS12 | SEQ ID NO: 12 | 124.2 ± 4.9 | — | — | — |
| CyPPS13 | SEQ ID NO: 13 | 422.4 ± 18.5 | 173.7 ± 13.0 | 7 ± 13 | 5 ± 2 |
| CyPPS14 | SEQ ID NO: 14 | 387.9 ± 2.7 | 231.4 ± 31.7 | 0 ± 10 | 2 ± 1 |
| CyPPS15 | SEQ ID NO: 15 | 280.5 ± 28.9 | — | — | — |
| CyPPS16 | SEQ ID NO: 16 | 198.0 ± 31.0 | — | — | — |
| CyPPS17 | SEQ ID NO: 17 | 203.0 ± 10.3 | — | — | — |
| CyPPS18 | SEQ ID NO: 18 | 245.7 ± 34.6 | — | — | — |
| CyPPS19 | SEQ ID NO: 19 | 286.8 ± 45.7 | — | — | — |
| CyPPS20 | SEQ ID NO: 20 | 282.7 ± 44.7 | — | — | — |
| CyPPS21 | SEQ ID NO: 21 | 278.2 ± 22.6 | — | — | — |
| CyPPS22 | SEQ ID NO: 22 | 297.5 ± 46.6 | — | — | — |
| CyPPS23 | SEQ ID NO: 23 | 352.4 ± 39.1 | 247.3 ± 15.4 | 0 ± 34 | 20 ± 4 |
| CyPPS24 | SEQ ID NO: 24 | 369.5 ± 22.6 | — | — | — |
| CyPPS25 | SEQ ID NO: 25 | 297.3 ± 10.4 | — | — | — |
| CyPPS27 | SEQ ID NO: 27 | 108.9 ± 4.1 | — | — | — |
| CyPPS28 | SEQ ID NO: 28 | 289.2 ± 11.0 | — | — | — |
| CyPPS29 | SEQ ID NO: 29 | 151.6 ± 5.8 | — | — | — |
| CyPPS30 | SEQ ID NO: 30 | 211.5 ± 12.5 | — | — | — |
| CyPPS31 | SEQ ID NO: 31 | 198.9 ± 8.6 | — | — | — |
| CyPPS32 | SEQ ID NO: 32 | 242.3 ± 21.8 | — | — | — |
| CyPPS33 | SEQ ID NO: 33 | 237.5 ± 7.7 | — | — | — |
| CyPPS34 | SEQ ID NO: 34 | 126.2 ± 9.6 | — | — | — |
| CyPPS35 | SEQ ID NO: 35 | 259.4 ± 11.4 | — | — | — |
| CyPPS36 | SEQ ID NO: 36 | 185.4 ± 5.7 | — | — | — |
| PPS1 | Linear peptide | 126.5 ± 4.8 | 83.7 ± 4.1 | 0 ± 18 | 0 ± 0 |
| | of CyPPS1 | | | | |

### Cell-Based Activity

While many cyclic peptides are cell-permeable, it is challenging to predict a priori the peptides that are capable of entering cells. Because the proteasome is an intracellular target, the ability of the CyPPS to enter cells was investigated. A BODIPY-tagged CyPPS was synthesized from the alkyne derivative CyPPS14 (see Scheme 2). A549 cells were then dosed with CyPPS26 (CyPPS14-attached with BODIPY™) and analyzed for cell permeability via confocal microscopy. Furthermore, investigated was the mechanism by which CyPPS26 was entering the cell. Cellular uptake of cyclic peptides usually occurs via passive diffusion or endocytosis. The puncta present suggests that CyPPS likely enters the cell via endosomal uptake. Endosomal uptake was confirmed by the overlap of the BODIPY™ and LysoTracker signal (FIG. 5A). The cytosolic fluorescence observed suggested that CyPPS is capable of escaping the endosome and thus likely engages with the cytosolic proteasome.

### EXAMPLES

Scheme 1 illustrates a method for preparing cyclic peptides based on the method reported in ACS Chemical Biology, 2021, 16 (11), 2604-2611, which is hereby specifically incorporated by reference for its teachings regarding the same.

### a) Resin Loading

Resin loading was carried out based on a method illustrated in Scheme 1. 2-Chlorotrityl chloride (2-CTC) resin (1 g, 0.77 mmol/g, 100-200 mesh), was swelled with DMF for 5 minutes. DMF was removed, and 2-CTC resin was treated with Fmoc-protected amino acid (3 equivalents), DIPEA (4 equivalents), and DMF (0.07 mM) for 2 hours. The solution was drained. A 17:2:1 solution of DCM: MeOH: DIPEA (0.07 mM) was combined with the 2-CTC resin and agitated for 30 minutes to cap the remaining unloaded resin. The solution was drained. The resin was filtered and washed with DCM (3×5 mL) and MeOH (3×5 mL) and dried for 1 hour. 1.5-3.0 mg of dried resin was subjected to 20% mixture of piperdine/DMF (500 μL) for 15 minutes. 100 μL of the supernatant was added to 900 μL of DMF three separate times. These were observed under UV absorbance at 301 nm.

### b) Solid Phase Peptide Synthesis

Solid phase peptide synthesis (SPPS) was carried out based on a method described in Scheme 1. Linear peptides were synthesized on a 0.05 mmol scale using a PS3 peptide synthesizer (Gyros Protein Technologies). Preloaded 2-chlorotrityl chloride resin (as described under resin loading) was utilized. Fmoc deprotection was achieved with 20% piperidine/DMF (2×5 minutes). DIC, Oxyma Pure, and Fmoc-protected amino acid (6 equivalents each) were utilized in a 1-hour coupling. The coupling and deprotection were repeated until the desired linear peptide was obtained.

### c) Resin Cleavage

Resin cleavage was carried out based on a method described in Scheme 1. The resin obtained via SPPS was subjected to DMF for 15 minutes to swell the resin in a 5 mL fritted polypropylene syringe (Torviq). The DMF was drained from the reaction vessel and treated with 20% piperidine/DMF for 15 minutes. The resin was washed with DMF (3×2 mL) and DCM (3×2 mL) and then dried. To verify a successful Fmoc deprotection, and Kaiser ninhydrin test was utilized. The successfully deprotected peptide was exposed to 25% HFIP/DCM for 30 minutes. The solution was concentrated in vacuo. The resulting residue was resuspended in 50% H₂O/MeCN, frozen, and lyophilized. The crude compound was used without further purification.

### d) Peptide Cyclization

Peptide cyclization was carried out based on a method described in Scheme 1. The crude peptides (˜0.05 mmol), PyBop (3 equivalents), DIPEA (6 equivalents), and DMF (1.25 mM) were agitated overnight (16-24 hours) and concentrated in vacuo. 50% H₂O/MeCN (10 mL) was added to the concentrated crude residue, vortexed, and centrifuged to obtain the precipitate and decant the supernatant. The resulting precipitate was washed with an additional 10 mL of 50% H₂O/MeCN, frozen, and lyophilized. If the residue did not crash out, a prep HPLC was utilized to afford the cyclized product.

### e) Global Deprotection

Global deprotection was carried out based on a method described in Scheme 1. The crude cyclized product was subjected to TFA: DCM: TIPS (50:45: 5, 20 mM) for 2 hours. The volatiles were removed with air. 2 mL of TBME was utilized to precipitate the peptides. The precipitate was vortexed and centrifuged to afford the precipitate that was then washed with additional TBME, dissolved in H₂O: MeCN, frozen, and lyophilized.

### Assays

### General:

HPLC analysis and purification was done using an Agilent Technologies 1260 Infinity II preparative HPLC system. Purity analysis was performed with a Luna C18 reverse phase 5 μm, 150×4.6 mm column. Purification was done with a Luna C18 reverse phase 5 μm, 150×21.2 mm column.

### HPLC Analysis

HPLC analysis was carried out based on a method reported in ACS Chemical Biology, 2021,16 (11), 2604-2611. Eluent A: H₂O with 0.1% FA. Eluent B: MeCN with 0.1% FA. Purity analysis utilized the gradient as follows: (A: B, 1 mL/min): 95:5, 0 min; 95:5, 1 min; 5:95, 20 min; 5:95,25 min; 95:5, 30 min. For purification, the gradient was as follows: (A: B, 20 mL/min) 95:5, 0 min; 95:5, 1 min; 5:95, 20 min; 5:95, 25 min; 95:5, 30 min.

### TAS-1 Assay

TAS-1 assay was carried out based on a method reported in ACS Combinatorial Science, 2018,20 (5), 269-276. Each compound was initially tested at 10 μM. Each well contained 154 μL of 11.4 μM TAS-1 solution in Tris HCl (50 mM, pH 7.4) along with 5 nM of 20 sCP (South Bay). 1 μL of the test compound, control, or DMSO was added to the desired well to obtain a final DMSO concentration of 2%, with the total volume being 50 μL. Compounds were tested in three replicates that each had technical triplicates. The fluorescence was recorded using a SynergyNeo 2, where the excitation and emission was set to 335 nm and 493 nm. The plate reader was heated to 37° C. The fluorescence was recorded every 90 seconds over a 1-hour period. A simple linear regression was performed on the data plotted against time to obtain the rate of hydrolysis of TAS-1 by the 20S CP. The data was then normalized against DMSO to get the percent stimulation.

### Hemolysis Assay

Hemolysis assay was carried out based on a method reported in ChemBioChem 2012, 13 (4), 574-583. Whole human blood was purchased at BiolVT and used prior to its expiration. 100 μL blood was added to a 1.5 mL Eppendorf tub with 500 μL of sterile 0.9% NaCl. The tube was inverted multiple times to mix the solution. This mixture was centrifuged at 500 g for 7 minutes. The supernatant was extracted utilizing a pipette. The pellet was washed 2 more times. After the final wash, 800 μL of Red Blood Cell (RBC) buffer was added to the pellet to resuspend the red blood cells. 4 μL of the compounds (20 μM in DMSO, final concentration), 76 μL RBC buffer, and 40 μL resuspended red blood cells were added to a 96 U-well plate (VWR) and incubated for 1 hour at 37° C. The plate was spun at 500 g for 5 minutes. 75 L of the supernatant was transferred to a 96 flat-well plate. The absorbance was read at 540 nm utilizing a SpectraMax iD3 platereader. Percent hemolysis was measured from the average absorbance of the positive and negative controls. A minimum of 3 biological triplicates were performed.

### SRB Cytotoxicity Assay

SRB cytotoxicity assay was carried out based on a method reported in Nature Protocols, 2006, 1(3), 1112-1116. A549 cells were obtained directly from ATCC and used within 20 passages. The cells were maintained using RPMI media with 10% Fetal Bovine Serum (FBS) and 1% streptomycin/penicillin. Cells were seeded at 5,000 cells per well, adhering overnight. They were treated at the desired concentration (1% DMSO final) for 72 hours. Viability was tested utilizing Sulfurhodamide B (SRB) and incubated at 5° C. for one hour. The absorbance was taken at 510 nm.

### Purified Protein Degradation Assay

Proteins were diluted to 50 ng/μL in Tris HCl (50 mM, pH 7.6). The CyPPS were diluted to 30 μM in Tris HCl, with 6% DMSO. The 20S CP (South Bay) was diluted to 15 nM with Tris HCl. Each protein was performed with a latent level, basal level, and CyPPS in triplicate. The latent level was prepared with 4 μL protein and 8 μL of 3% DMSO in Tris HCl. The basal level contained 4 μL protein, 4 μL 20S CP, and 4 μL 6% DMSO in Tris HCl. The CyPPS was performed with 4 μL protein, 4 μL 20S CP, and 4 μL 30 μM CyPPS. The samples were incubated for 1 hour at 37° C. 4 μL of 4×SDS gel loading buffer was added and heated for 5 minutes at 95° C. The samples were run on a 15-well gradient gel for SDS-PAGE, stained with Coomassie, imaged with an imaging system, and quantified with ImageJ. Analysis was done via GraphPad PRISM9.

### Fluorophore Conjugation

Fluorophore conjugation was prepared based on a method described in Organic letters, 2011, 13(20), 5656-5659. BODIPY™ FL Azide (6.68 μmol) was dissolved in 80 μL of DMSO. CyPPS14 (7.35 μmol) was dissolved in 500 μL tert-butanol. The solution was degassed under argon. Ascorbate (6.66 μmol, aqueous solution, 80 μL) and copper sulfate (0.68 μmol, aqueous solution, 80 μL) were added to the mixture and heated for 1 hour at 80° C. HPLC was performed to analyze reaction purity and MS was utilized to analyze completion. Purification was carried out via an HPLC.

### Confocal Imaging

A549 cells were obtained from ATCC and used within 20 passages. The cells were maintained using RPMI media with 10% FBS and 1% streptomycin/penicillin. Cells were seeded at 950,000 cells per well, adhering overnight. They were treated with CyPPS26 (15 μM) and LysoTracker (75 μM) (<1% DMSO) for 1.5 hours. Treated cells were washed three times with phosphate-buffered saline (PBS) and 2.5% Hoescht 33342: Krebs-Ringer Bicarbonate Buffer (KRBH) was incubated for 30 minutes. Imaging was performed using a Nikon A1Rsi Confocal.

### Flow Cytometry

Confluent HEK 293T cells were seeded into sterilized 2 mL Fisherbrand Eppendorf tubes in FBS free Dulbecco's Modified Eagle Medium (DMEM) at a concentration of 300,000 cells per tube. The cells were centrifuged at 1000 g for 5 minutes. The supernatant was removed, and the cells were resuspended in 500 μL of a 10 μM stimulator solution in FBS free DMEM. Cells were spun down at 1000 g for 5 minutes, and the supernatant was again removed. Cells were then resuspended in 500 μL of a 10 μM stimulator 2.5 μM epoxomicin probe in FBS-free DMEM and incubated for 1 hour at 37° C. Cells were again centrifuged at 1000 g for 5 minutes. Supernatant was then removed, and cells were washed once with cold PBS. Cells were then resuspended in 50 μL of cold PBS and placed on ice. Cellular fluorescence was measured by collecting 50,000 events using the FITC channel of a BD Accuri C6 Flow Cytometer and analyzed using FlowJo.

### Epoxomicin Probe Synthesis

Synthesis was performed using the procedure described in art (see Current Protocols, 2022, 2(7), e490).

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid the reading of the document and is not to be interpreted as limiting. Further, information that is relevant to a section heading may occur within or outside of that particular section. The terms “including” and “having” are defined as comprising (i.e., open language).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

We claim:

1. A pharmaceutical composition comprising:
(a) a cyclic peptide of formula (I): 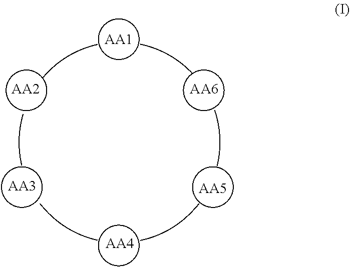
wherein each of AA1, AA2, AA3, AA4, AA5, and AA6 is independently a natural amino acid or an unnatural amino acid with the proviso that formula (I) comprises at least one of each of an aromatic amino acid, a polar amino acid, and an arginine;
and (b) a pharmaceutically acceptable carrier.

2. The pharmaceutical composition of claim 1, wherein the natural amino acid or unnatural amino acid is selected from threonine, leucine, phenylalanine, arginine, lysine, aspartic acid, valine, isoleucine, propargylglycine, 3-fluorophenylalanine, 3,4-difluorophenylalanine, tyrosine, 4-benzoylphenylalanine, serine, alanine, 4-fluorophenylalanine, tryptophan, diaminopropionic acid, and diaminobutyric acid.

3. The pharmaceutical composition of claim 1, wherein the cyclic peptide is CyPPS1, a cyclic peptide of formula (II): 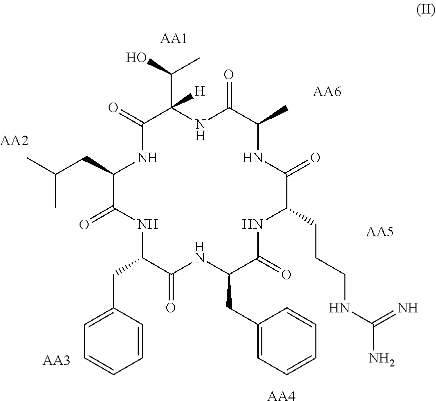
wherein AA1 is Thr, AA2 is D-Leu, AA3 is Phe, AA4 is D-Phe, AA5 is Arg, and AA6 is D-Ala comprising a sequence of [SEQ ID NO: 1] or a derivative thereof selected from: [SEQ ID NO: 2]-[SEQ ID NO: 36].

4. The pharmaceutical composition of claim 1, further comprising one or more additional therapeutic agents.

5. A method of increasing protein degradation by the proteasome system in a patient with a proteinopathy, which method comprises administering a therapeutically effective amount of a pharmaceutical composition of claim 1.

6. The method of claim 5, wherein the patient has a prion (misfolded protein) disease, Creutzfeldt-Jakob disease (neurocognitive disorder due to prion disease), Alzheimer's disease, Parkinson's disease, amyloidosis, multiple system atrophy, or Huntington's disease.

7. A cyclic peptide selected from [SEQ ID NO: 2]-[SEQ ID NO: 36].
